(19) United States
(12) Patent Application Publication
(10) Pub. No.: US 2026/0174368 A1
(43) Pub. Date: Jun. 25, 2026

(54) BEHAVIOR PREDICTION DEVICE, BEHAVIOR PREDICTION METHOD, AND BEHAVIOR PREDICTION PROGRAM

(71) Applicant: NTT, Inc., Tokyo (JP)

(72) Inventors: Yu ADACHI, Tokyo (JP); Nagisa SEKIGUCHI, Tokyo (JP); Mina KATAGIRI, Tokyo (JP)

(73) Assignee: NTT, Inc., Tokyo (JP)

(21) Appl. No.: 19/127,691

(22) PCT Filed: Nov. 11, 2022
(86) PCT No.: PCT/JP2022/042149

§ 371 Date: May 6, 2025

(51) Int. Cl. A61B 5/16; G06N 7/01

(52) U.S. Cl. CPC A61B 5/165; G06N 7/01

(57) ABSTRACT

A behavior prediction device is a behavior prediction device that predicts future behavior of a specific person, the behavior prediction device including: an information extraction unit that extracts start time probability distribution data indicating a probability distribution of a start time for each type of behavior, behavior transition probability data indicating a probability of transition from one behavior to the other behavior for each combination of the types of behaviors, and behavioral time data regarding a behavioral time of the each type of behavior on the basis of a series of past behaviors indicating a past behavior history of the specific person in chronological order; and a behavior prediction unit that predicts the future behavior of the specific person on the basis of the start time probability distribution data, the behavior transition probability data, the behavioral time data, and a current behavior of the specific person.

### TECHNICAL FIELD

The disclosed technology relates to a behavior prediction device, a behavior prediction method, and a behavior prediction program.

### BACKGROUND ART

Technology for predicting a person's future behavior has been conventionally known. For example, Patent Literature 1 discloses a technology of predicting a user's behavior on the basis of the user's place of stay, the user's time of stay, and a probability of appearance of each past behavior pattern.

### CITATION LIST

### Patent Literature

Patent Literature 1: JP 2010-250759 A

### SUMMARY OF INVENTION

### Technical Problem

However, in the technology disclosed in Patent Literature 1, a behavior information pattern indicating a start time of behavior, an end time of the behavior, and transition behavior in a place of stay is used as a factor that determines the next behavior. However, only the factor disclosed in Patent Literature 1 is not sufficient as a factor that predicts future behavior, Therefore, accuracy of predicting a person's behavior can be further improved.

The disclosed technology has been made in view of the above points, and an object thereof is to provide a behavior prediction device, a behavior prediction method, and a behavior prediction program capable of predicting future behavior of a specific person with higher accuracy.

### Solution to Problem

A first aspect of the present disclosure is a behavior prediction device that predicts future behavior of a specific person, the behavior prediction device including: an information extraction unit that extracts start time probability distribution data indicating a probability distribution of a start time for each type of behavior, behavior transition probability data indicating a probability of transition from one behavior to the other behavior for each combination of the types of behaviors, and behavioral time data regarding a behavioral time of the each type of behavior on the basis of a series of past behaviors indicating a past behavior history of the specific person in chronological order; and a behavior prediction unit that predicts the future behavior of the specific person on the basis of the start time probability distribution data, the behavior transition probability data, the behavioral time data, and a current behavior of the specific person.

A second aspect of the present disclosure is a behavior prediction method of predicting future behavior of a specific person, in which: an information extraction unit extracts start time probability distribution data indicating a probability distribution of a start time for each type of behavior, behavior transition probability data indicating a probability of transition from one behavior to the other behavior for each combination of the types of behaviors, and behavioral time data regarding a behavioral time of the each type of behavior on the basis of a series of past behaviors indicating a past behavior history of the specific person in chronological order; and a behavior prediction unit predicts the future behavior of the specific person on the basis of the start time probability distribution data, the behavior transition probability data, the behavioral time data, and a current behavior of the specific person.

A third aspect of the present disclosure is a behavior prediction program, which is a program for causing a computer to function as each unit of the behavior prediction device of the above first aspect.

### Advantageous Effects of Invention

According to the disclosed technology, it is possible to predict future behavior of a specific person with higher accuracy.

### BRIEF DESCRIPTION OF DRAWINGS

FIG. 1 is a configuration diagram showing an example of a configuration of a behavior prediction system according to an embodiment.

FIG. 2 is a configuration diagram showing an example of a hardware configuration of the behavior prediction device according to the embodiment.

FIG. 3 is a block diagram showing an example of a functional configuration of the behavior prediction device according to the embodiment.

FIG. 4 shows a transition example of behavior of a specific person for two weeks,

FIG. 5A shows an example of behavior transition probability data.

FIG. 5B shows an example of elapsed time probability distribution data.

FIG. 5C shows an example of start time probability distribution data.

FIG. 5D shows an example of end time probability distribution data.

FIG. 5E shows an example of behavioral time probability distribution data.

FIG. 6 is an explanatory diagram showing an operation of an occurring behavior/behavior start time prediction unit.

FIG. 7 is an explanatory diagram showing an operation of a behavior end time prediction unit.

FIG. 8 is a flowchart showing an example of information extraction processing in the behavior prediction device according to the embodiment.

FIG. 9 is a flowchart showing an example of behavior prediction processing in the behavior prediction device according to the embodiment.

### DESCRIPTION OF EMBODIMENTS

An example of an embodiment of the disclosed technology will be described below with reference to the drawings. In the drawings, the same or equivalent components and portions are denoted by the same reference signs, Dimensional ratios in the drawings are exaggerated for convenience of description and thus may be different from actual ratios.

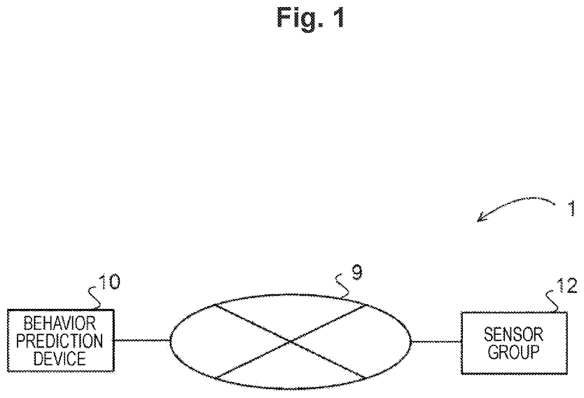

First, an example of a configuration of a behavior prediction system 1 according to the technology of the present embodiment will be described. As shown in FIG. 1, the behavior prediction system 1 of the present embodiment includes a behavior prediction device 10 and a sensor group 12. The behavior prediction device 10 and the sensor group 12 are connected by wired communication or wireless communication via a network 9.

The sensor group 12 includes a plurality types of sensors used for detecting an event that changes depending on a person's behavior. Specific types of sensors are different for each event that changes depending on a person's behavior to be detected, and examples thereof include a sensor that detects any of vital signs of a person, a place where the person stays, a temperature, humidity, illuminance, volume, a sleeping state of the person, a wakefulness state of the person, and an amount of electric consumption. A detection result of each sensor included in the sensor group 12 is output to the behavior prediction device 10 via the network 9.

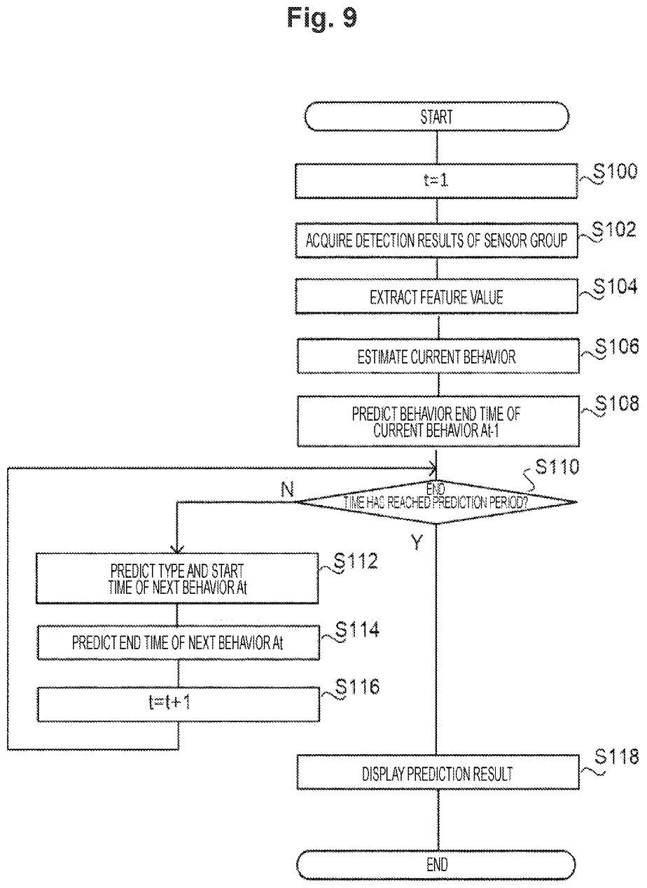

The behavior prediction device 10 is a device that predicts future behavior of a specific person. The behavior prediction device 10 of the present embodiment estimates a current behavior of the specific person on the basis of a detection result of an event that changes depending on the specific person's behavior input from the sensor group 12. Further, the behavior prediction device 10 predicts the future behavior of the specific person on the basis of statistical information extracted from a series of past behaviors indicating a past behavior history of the specific person in chronological order and the current behavior. The “current behavior” refers to a behavior at a start time of a prediction period which is a start point for predicting the future behavior of the specific person. Therefore, the “current behavior” may be different from a behavior at a time when behavior prediction processing (see FIG. 9) described below is executed.

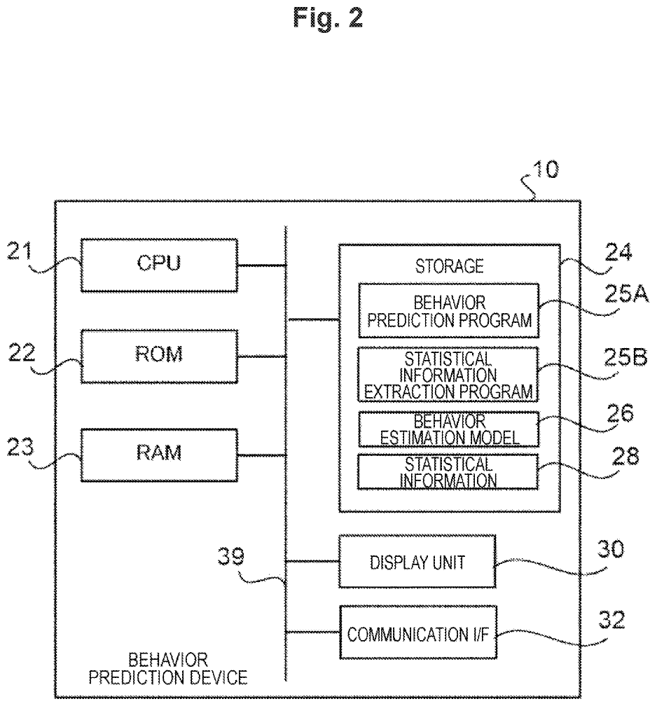

FIG. 2 is a configuration diagram showing an example of a hardware configuration of the behavior prediction device 10 according to the present embodiment. As shown in FIG. 2, the behavior prediction device 10 includes a central processing unit (CPU) 21, a read only memory (ROM) 22, a random access memory (RAM) 23, a storage 24, a display unit 30, and a communication interface (I/F) 32. The components are communicably connected to each other via a bus 39 such as a system bus or a control bus.

The CPU 21 is a central processing unit and executes various programs such as a behavior prediction program 25A stored in the storage 24 and controls each unit.

The ROM 22 stores various programs executed by the CPU 21 and various types of data. The RAM 23 temporarily stores programs or data as a working area when the CPU 21 executes various programs. That is, the CPU 21 reads a program from the storage 24 and executes the program by using the RAM 23 as a working area.

The storage 24 of the present embodiment stores the behavior prediction program 25A and a statistical information extraction program 25B. Note that the behavior prediction program 25A and the statistical information extraction program 25B each may be one program or a program group including a plurality of programs or modules. The storage 24 includes a hard disk drive (HDD) or a solid state drive (SSD). Further, the storage 24 stores various programs including an operating system and various types of data (both not shown). The storage 24 further stores a behavior estimation model 26 used for estimating a person's behavior.

The display unit 30 displays various types of information such as information regarding the future behavior of the specific person as a prediction result. The display unit 30 is not particularly limited, and examples thereof include various displays.

The communication I/F 32 is an interface for communicating with the sensor group 12 via the network 9 and adopts a standard such as Ethernet (registered trademark), FDDI, or Wi-Fi (registered trademark).

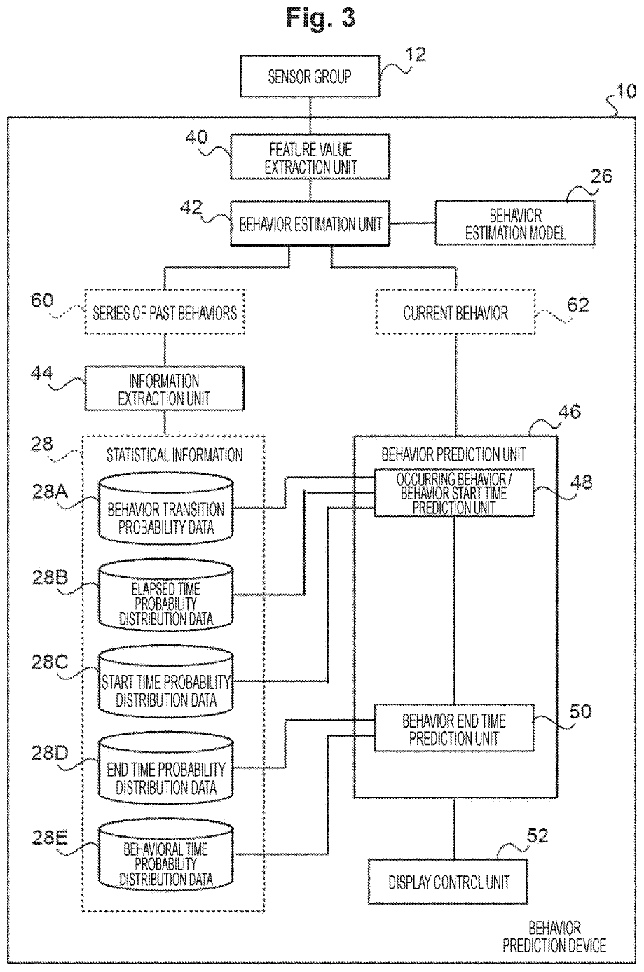

Next, a functional configuration of the behavior prediction device 10 will be described. As shown in FIG. 3, the behavior prediction device 10 includes a feature value extraction unit 40, a behavior estimation unit 42, an information extraction unit 44, a behavior prediction unit 46, and a display control unit 52. When the CPU21 executes the statistical information extraction program 25B stored in the storage 24, the CPU21 functions as the feature value extraction unit 40, the behavior estimation unit 42, and the information extraction unit 44. When the CPU21 executes the behavior prediction program 25A stored in the storage 24, the CPU21 functions as the feature value extraction unit 40, the behavior estimation unit 42, the behavior prediction unit 46, and the display control unit 52.

The feature value extraction unit 40 has a function of extracting a feature value regarding an event that changes depending on a person's behavior from detection results input from the sensor group 12. The feature value to be extracted corresponds to the person's behavior, and examples thereof include vital signs of the person, a place where the person stays, a temperature, humidity, illuminance, volume, a sleeping state of the person, a wakefulness state of the person, and an amount of electric consumption. The feature value extraction unit 40 outputs the extracted feature value to the behavior estimation unit 42.

The behavior estimation unit 42 has a function of deriving the type of behavior (a series of past behaviors 60 or a current behavior 62) of the specific person on the basis of the feature value input from the feature value extraction unit 40 by using the behavior estimation model 26.

The behavior estimation model 26 is a learned model that receives the feature value extracted by the feature value extraction unit 40 as an input and outputs the type of behavior. Examples of the type of behavior include waking up, getting dressed, cooking, eating, working, doing leisure activities, and sleeping, but are not limited thereto.

As an example, the behavior estimation model 26 of the present embodiment is a model based on a decision tree. The behavior estimation model 26 is obtained, for example, by learning a learning model in the following learning phase. The behavior estimation model 26 is learned by being given learning data, which is also referred to as training data, in the learning phase. The learning data is a set of the feature value extracted by the feature value extraction unit 40 and the type of behavior that is a correct answer. In the learning phase, the feature value extracted by the feature value extraction unit 40 is vectorized and input to the behavior estimation model 26. The behavior estimation model 26 outputs the type of behavior as an estimation result for the feature value extracted by the feature value extraction unit 40. The behavior estimation model 26 is learned by optimizing each branch of the decision tree on the basis of the type of behavior that is the estimation result and the type of behavior that is the correct answer.

As in the present embodiment, the behavior prediction device 10 may acquire the behavior estimation model 26 learned by an external device and store the behavior estimation model in the storage 24. Unlike the present embodiment, the behavior prediction device 10 may learn the behavior estimation model 26. Further, unlike the present embodiment, the behavior prediction device 10 may not store the behavior estimation model 26, and the behavior estimation unit 42 may use the behavior estimation model 26 stored in the external device.

The behavior estimation model 26 may be a model specialized for each person or a general-purpose model. Specifically, the behavior estimation model 26 may be specialized for a specific person's behavior by using, as the learning data, only a feature value extracted by the feature value extraction unit 40 from detection results of the sensor group 12 regarding the specific person. The behavior estimation model may also be specialized for general behavior of people by using, as the learning data, feature values extracted by the feature value extraction unit 40 from detection results of the sensor group 12 regarding a plurality of people.

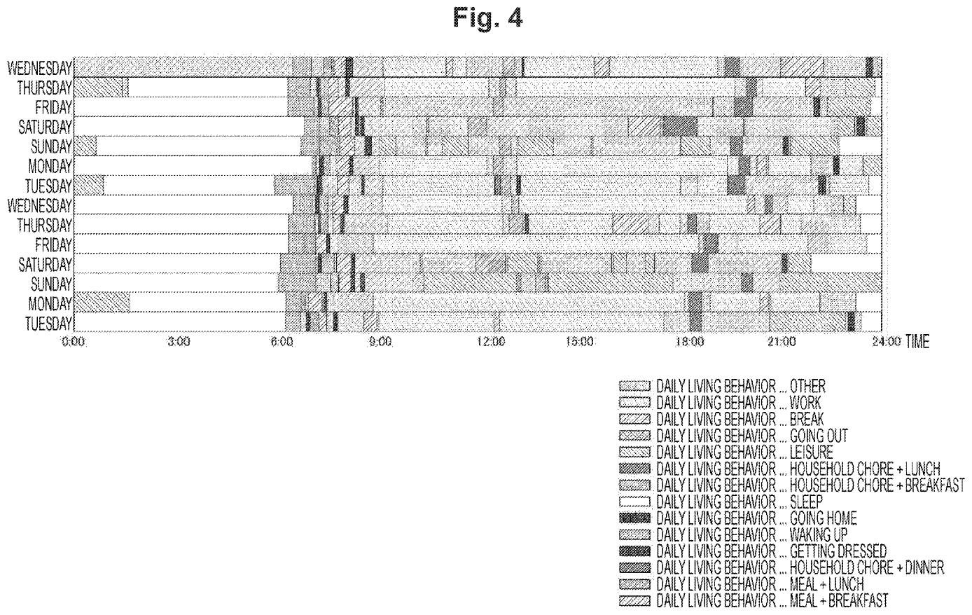

The behavior estimation unit 42 inputs the feature value input from the feature value extraction unit 40 to the behavior estimation model 26 and acquires the type of behavior of the person output from the behavior estimation model 26. Note that, in a case of generating statistical information 28 regarding the specific person's behavior, the behavior estimation unit 42 outputs the series of past behaviors 60 indicating the types of behaviors of the specific person in chronological order to the information extraction unit 44. FIG. 4 shows, as an example, transition of the specific person's behavior for two weeks. In this case, the behavior estimation unit 42 outputs the series of past behaviors 60 indicating the types of behaviors according to the transition example in FIG. 4 in chronological order to the information extraction unit 44. As described above, the series of past behaviors 60 indicates the past behavior history of the specific person in chronological order.

In a case of generating the statistical information 28 regarding the specific person's behavior, the series of past behaviors 60 is input from the behavior estimation unit 42 to the information extraction unit 44, The information extraction unit 44 has a function of extracting the statistical information 28 from the series of past behaviors 60. In other words, the information extraction unit 44 has a function of generating the statistical information 28 by using the series of past behaviors 60 as the learning data. As shown in FIG. 3, the statistical information 28 includes behavior transition probability data 28A, elapsed time probability distribution data 28B, start time probability distribution data 28C, end time probability distribution data 28D, and behavioral time probability distribution data 28E.

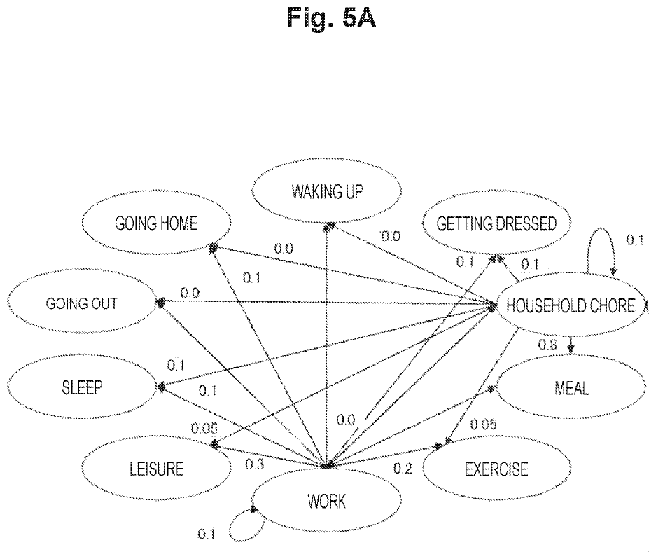

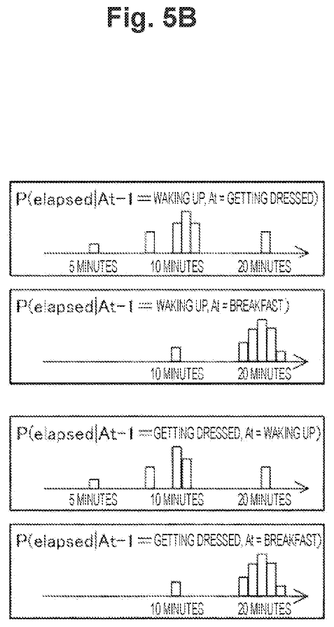

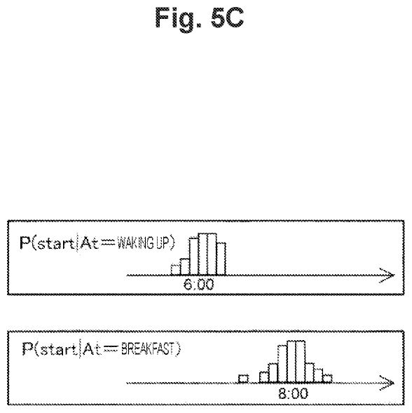

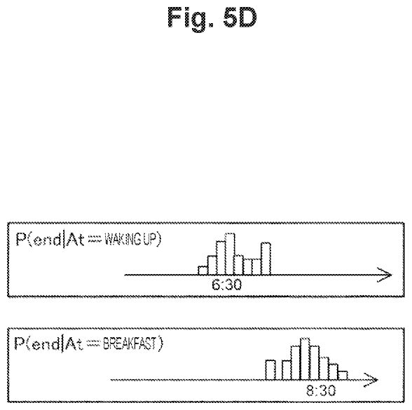

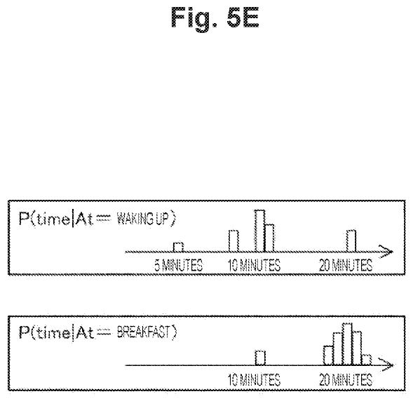

The behavior transition probability data 28A is data indicating a probability of transition (transition probability P(At−1→At)) from one behavior (behavior At−1) to the other behavior (behavior At) for each combination of the types of behaviors. FIG. 5A shows an example of the behavior transition probability data 28A. As shown in FIG. 5A, the behavior transition probability data 28A of the present embodiment expresses transition behaviors from a certain behavior to the next behavior as a Markov model. Note that FIG. 5A shows the types of behaviors and some transition behaviors, but the actual behavior transition probability data 28A indicates transition behaviors for all combinations of the types of behaviors to be estimated.

The elapsed time probability distribution data 28B is data indicating a probability distribution of the elapsed time from the start of one behavior (behavior At−1) to the start of the other behavior (behavior At) for each combination of the types of behaviors. FIG. 5B shows an example of the elapsed time probability distribution data 28B. A behavioral time during which a certain behavior (behavior At−1) is performed may be different for each type of the next behavior (behavior At). For example, as shown in FIG. 5B, a behavioral time during which the behavior of waking up is performed in a case where the behavior of getting dressed is performed after the behavior of waking up may be different from a behavioral time during which the behavior of waking up is performed in a case where the behavior of eating breakfast is performed after the behavior of waking up. Specifically, the elapsed time from the start of waking up to the start of getting dressed that is the next behavior may be different from the elapsed time from the start of waking up to the start of eating breakfast that is the next behavior.

Further, for example, as shown in FIG. 5B, a behavioral time during which the behavior of getting dressed is performed in a case where the behavior of waking up is performed after the behavior of getting dressed may be different from a behavioral time during which the behavior of getting dressed is performed in a case where the behavior of eating breakfast is performed after the behavior of getting dressed. Specifically, the elapsed time from the start of getting dressed to the start of waking up that is the next behavior may be different from the elapsed time from the start of getting dressed to the start of eating breakfast that is the next behavior.

Therefore, the information extraction unit 44 of the present embodiment extracts, as the elapsed time probability distribution data 28B, data indicating a probability distribution of the elapsed time from the start of one behavior (behavior At−1) to the start of the other behavior (behavior At) for each combination of the types of behaviors. As described above, the elapsed time probability distribution data 28B is data indicating tendency of the behavioral time according to the type of the next behavior (behavior At).

The start time probability distribution data 28C is data indicating a probability distribution of the start time of each behavior (behavior At). FIG. 5C shows an example of the start time probability distribution data 28C. Note that FIG. 5C shows the probability distribution of the start time in a case where the type of behavior is waking up and the probability distribution of the start time in a case where the type of behavior is eating breakfast.

The end time probability distribution data 28D is data indicating a probability distribution of the end time of each type of behavior. FIG. 5D shows an example of the end time probability distribution data 28D. Note that FIG. 5D shows the probability distribution of the end time in a case where the type of behavior is waking up and the probability distribution of the end time in a case where the type of behavior is eating breakfast.

The behavioral time probability distribution data 28E is data indicating a probability distribution of the behavioral time during which behavior is performed for each type of behavior. FIG. 5E shows an example of the behavioral time probability distribution data 28E. Note that FIG. 5E shows the probability distribution of the behavioral time in a case where the type of behavior is waking up and the probability distribution of the behavioral time in a case where the type of behavior is eating breakfast.

The information extraction unit 44 stores the extracted statistical information 28 in the storage 24.

Meanwhile, in a case where the future behavior of the specific person is predicted, the behavior estimation unit 42 outputs the current behavior 62 indicating the type of the current behavior of the specific person to the behavior prediction unit 46. As shown in FIG. 3, the behavior prediction unit 46 includes an occurring behavior/behavior start time prediction unit 48 and a behavior end time prediction unit 50.

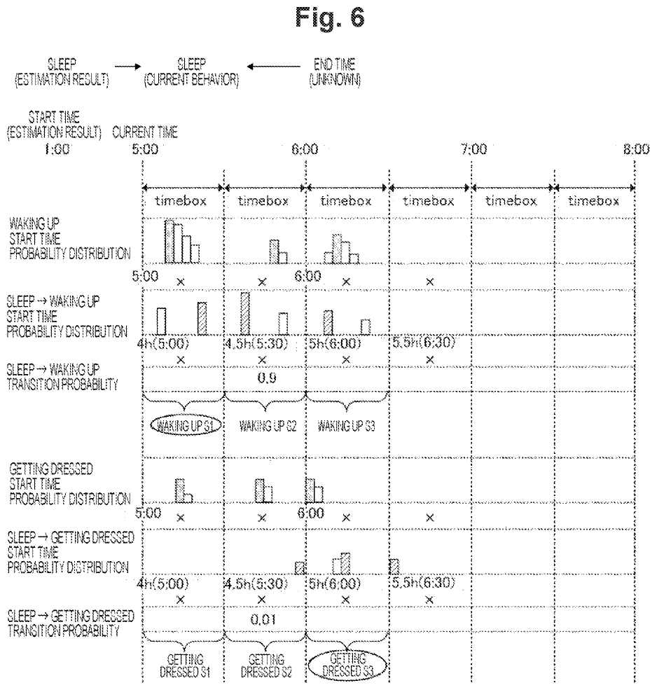

The occurring behavior/behavior start time prediction unit 48 has a function of predicting the type of the next behavior (behavior At) and the start time of the behavior At on the basis of the behavior transition probability data 28A, the elapsed time probability distribution data 28B, and the start time probability distribution data 28C. FIG. 6 shows an example of predicting the type of the next behavior (behavior At) after the behavior of sleeping (behavior At−1) and the start time of the behavior At. The occurring behavior/behavior start time prediction unit 48 derives a score S in which both the start time of the behavior and the elapsed time to the next behavior are considered for each type of behavior in the unit of timebox obtained by dividing time by a predetermined unit (30 minutes in FIG. 6) from Expression (1) below.

$$S = \{ Pmax ( start \vert At ) \} + Pmax ( elapsed \vert At - 1 \to At ) + P ( At - 1 \to At ) \qquad ( 1 )$$

Here, Pmax(start|At) represents a maximum probability in the timebox in the start time probability distribution, Pmax(elapsed|At−1→At) represents a maximum probability in the timebox in the elapsed time probability distribution, and P(At−1→At) represents the transition probability from the behavior At−1 to the behavior At.

In the example of FIG. 6, in a case where the type of behavior is waking up, a score S1 of a timebox from 5:00 to 5:30 is the highest, and thus the score S1 becomes the score S used for estimating a candidate for the next behavior At, and the timebox from 5:00 to 5:30 is selected. In a case where the type of behavior is getting dressed, a score S3 of a timebox from 6:00 to 6:30 is the highest, and thus the score S3 becomes the score S used for estimating a candidate for the next behavior At, and the timebox from 6:00 to 6:30 is selected.

The occurring behavior/behavior start time prediction unit 48 derives a candidate for the start time from Expression (2) below by using a timebox selected based on the score S for each type of behavior,

$$Start time ( candidate in behavior At ) = \{ start of Pmax ( start \vert At ) + ( start time of At - 1 + time of Pmax ( elapsed \vert At - 1 \to At ) \} \div 2 \qquad ( 2 )$$

Here, start of Pmax(start|At) represents a start time when the probability becomes maximum in the timebox in the start time probability distribution, and time of Pmax(elapsed|At−1→At) represents the elapsed time during which the probability becomes maximum in the timebox in the elapsed time probability distribution.

The occurring behavior/behavior start time prediction unit 48 compares the scores S of respective types of behaviors and estimates the type of behavior having the maximum score S as the next behavior At. For example, in the above example, because the score S1 of waking up>the score S3 of getting dressed is satisfied, waking up is estimated as the next behavior At, and the start time of waking up obtained from Expression (2) above is predicted as the start time of the next behavior At.

The occurring behavior/behavior start time prediction unit 48 outputs the predicted next behavior At and the start time thereof to the behavior end time prediction unit 50.

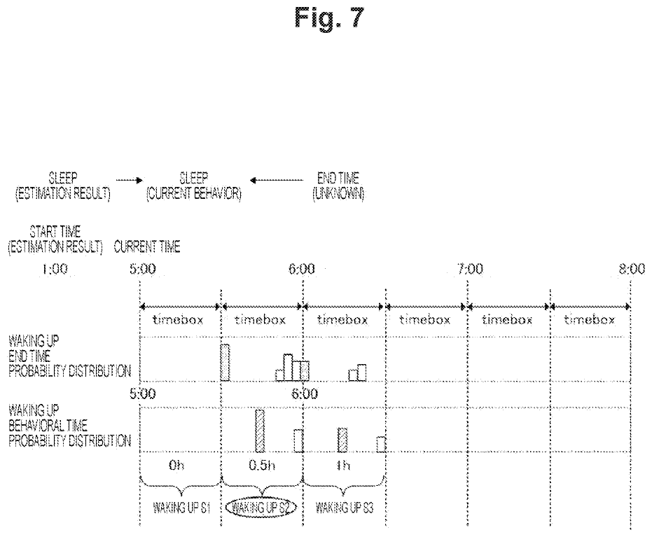

The behavior end time prediction unit 50 has a function of predicting the end time of the next behavior At estimated by the occurring behavior/behavior start time prediction unit 48 on the basis of the end time probability distribution data 28D and the behavioral time probability distribution data 28E. FIG. 7 shows an example of predicting the end time in a case where the next behavior At is waking up. The occurring behavior/behavior start time prediction unit 48 derives the score S in which both the end time and the behavioral time of the behavior are considered for each behavior in the unit of timebox obtained by dividing time by a predetermined unit (30 minutes in FIG. 7) from Expression (3) below.

$$S = \{ Pmax ( end \vert At ) \} + Pmax ( time \vert At ) \qquad ( 3 )$$

Here, Pmax(end|At) represents a maximum probability in the timebox in the end time probability distribution, and Pmax(time|At) represents a maximum probability in the timebox in the behavioral time probability distribution.

In the example of FIG. 7, in a case where the type of behavior is waking up, the score S2 is the highest, and thus 5:30 to 6:00 that is a timebox corresponding to waking up S2 is selected as a candidate for the end time.

For the estimated next behavior At, the behavior end time prediction unit 50 derives a candidate for the end time in the selected timebox from Expression (4) below.

$$End time ( candidate in behavior At ) = \{ end of Pmax ( end \vert At ) + ( start time of At + time of Pmax ( time \vert At ) \} \div 2 \qquad ( 4 )$$

Here, end of Pmax(end|At) represents the end time at which the probability becomes maximum in the timebox in the end time probability distribution, and time of Pmax (time|At) represents the behavioral time during which the probability becomes maximum in the timebox in the behavioral time probability distribution.

The behavior end time prediction unit 50 outputs the end time of the predicted next behavior At.

The behavior prediction unit 46 outputs the next behavior At predicted by the occurring behavior/behavior start time prediction unit 48, the start time thereof, and the end time of the next behavior At predicted by the behavior end time prediction unit 50 to the display control unit 52.

The display control unit 52 has a function of displaying the type of behavior, the start time, and the end time of future behavior of the specific person estimated by the behavior estimation unit 42 in chronological order on the display unit 30. Instead of or in addition to displaying the type of behavior, the start time, and the end time, those may be stored in the storage 24.

Next, an operation of the behavior prediction device 10 of the present embodiment will be described.

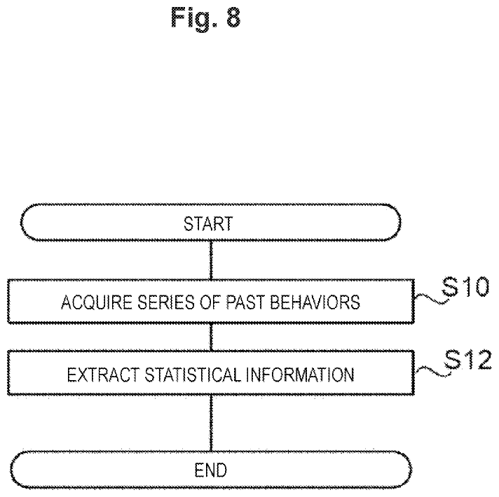

FIG. 8 is a flowchart showing an example of statistical information extraction processing executed by the behavior prediction device 10 according to the present embodiment. The behavior prediction device 10 executes the statistical information extraction processing in FIG. 8 by executing the statistical information extraction program 25B stored in the storage 24. Note that the statistical information extraction processing in FIG. 8 is executed at a predetermined timing such as a timing when an execution instruction from a user is received.

In step S10 of FIG. 8, the information extraction unit 44 acquires the series of past behaviors 60 as described above.

In the next step S12, the information extraction unit 44 extracts the behavior transition probability data 28A, the elapsed time probability distribution data 28B, the start time probability distribution data 28C, the end time probability distribution data 28D, and the behavioral time probability distribution data 28E as the statistical information 28 as described above. Note that methods of extracting the behavior transition probability data 28A, the elapsed time probability distribution data 28B, the start time probability distribution data 28C, the end time probability distribution data 28D, and the behavioral time probability distribution data 28E are not particularly limited, and known methods can be used. When the processing in step S12 ends, the statistical information extraction processing in FIG. 8 ends.

After the statistical information 28 is extracted in this manner, the behavior prediction device 10 executes behavior prediction processing in FIG. 9. The behavior prediction processing in FIG. 9 is processing for predicting behaviors A0, A1, A2, A3 . . . of a specific person from the current time to the end time of a prediction period. FIG. 9 is a flowchart showing an example of the behavior prediction processing executed by the behavior prediction device 10 according to the present embodiment. The behavior prediction device 10 executes the behavior prediction processing in FIG. 9 by executing the behavior prediction program 25A stored in the storage 24. Note that the behavior prediction processing in FIG. 9 may be executed after the statistical information extraction processing in FIG. 8 ends or may be executed at a timing when an execution instruction from the user is received after the statistical information extraction processing, i.e., is executed at a predetermined timing.

In step S100 of FIG. 9, the feature value extraction unit 40 sets a variable t to zero (t=0).

In the next step S102, the feature value extraction unit 40 acquires detection results of the sensor group 12 as described above.

In the next step S104, the feature value extraction unit 40 extracts a feature value regarding an event that changes depending on a person's behavior from a detection result input from the detection results of the sensor group 12 as described above.

In the next step S106, the behavior estimation unit 42 estimates a current behavior of a specific person. As described above, the behavior estimation unit 42 inputs the feature value extracted by the feature value extraction unit 40 to the behavior estimation model 26 and acquires the type of behavior output from the behavior estimation model 26 as the current behavior 62.

In the next step S108, the behavior end time prediction unit 50 of the behavior prediction unit 46 predicts the behavior end time of the current behavior At−1. Note that, because t=0 is satisfied as described above, the current behavior At−1=A0 is satisfied here. As described above, the behavior end time prediction unit 50 of the present embodiment selects a timebox having the largest score S as the end time of the current behavior At−1. Then, the behavior end time prediction unit 50 predicts the end time of the current behavior At−1 from Expression (5) below in which the “next behavior At” in Expression (4) above is changed to the current behavior “At−1”.

$$End time ( candidate in current behavior At - 1 ) = \{ end of Pmax ( end \vert At - 1 ) + ( start time of At - 1 + time of Pmax ( time \vert At - 1 ) \} \div 2 \qquad ( 5 )$$

In the next step S110, the occurring behavior/behavior start time prediction unit 48 determines whether or not the end time derived in the above step S108 has reached the end time of the prediction period. In the present embodiment, a prediction period for predicting future behavior of the specific person is set. For example, in a case where behaviors from the current time to 24 hours later are predicted, the prediction period is 24 hours. Note that the prediction period may be set in the behavior prediction device 10 in advance or may be settable by the user who performs prediction. Until the end time reaches the end time of the prediction period, a negative determination is made in step S110, and the processing proceeds to step S112.

In step S112, the occurring behavior/behavior start time prediction unit 48 predicts the type and start time of the next behavior At. As described above, the occurring behavior/behavior start time prediction unit 48 predicts the type of the next behavior At and the start time thereof from Expressions (1) and (2) above on the basis of the behavior transition probability data 28A, the elapsed time probability distribution data 28B, and the start time probability distribution data 28C.

In the next step S114, the behavior end time prediction unit 50 predicts the end time of the next behavior At predicted in the above step S112. As described above, the behavior end time prediction unit 50 predicts the end time of the next behavior At from Expressions (3) and (4) above on the basis of the end time probability distribution data 28D and the behavioral time probability distribution data 28E.

In the next step S116, the occurring behavior/behavior start time prediction unit 48 adds 1 to the variable t (t=t+1) as preparation for predicting a behavior after next, then returns to step S11, and repeats the processing in steps S110 to S116 until the end time reaches the end time of the prediction period. Therefore, prediction is sequentially performed for behaviors A2, A3, . . . .

Meanwhile, when the end time has reached the end time of the prediction period in step S110, a positive determination is made, and the processing proceeds to step S118.

In step S118, the display control unit 52 displays a prediction result on the display unit 30. As described above, the display control unit 52 displays the type of behavior, the start time, and the end time of future behavior of the specific person estimated by the behavior estimation unit 42 in chronological order on the display unit 30. When the processing in step S118 ends, the behavior prediction processing in FIG. 9 ends.

As described above, the behavior prediction device 10 of the present embodiment is a behavior prediction device that predicts future behavior of a specific person and includes the information extraction unit 44 and the behavior prediction unit 46.

Based on the series of past behaviors 60 in which the past behavior history of the specific person is indicated in chronological order, the information extraction unit 44 extracts the start time probability distribution data 28C indicating the probability distribution of the start time for each behavior type, the end time probability distribution data 28D indicating the probability distribution of the end time for each behavior type, the behavior transition probability data 28A indicating the probability of transition from one behavior to the other behavior for each combination of types of behavior, and the behavioral time probability distribution data 28E regarding the behavioral time for each behavior type. The behavior prediction unit 46 predicts the future behavior of the specific person on the basis of the start time probability distribution data 28C, the end time probability distribution data 28D, the behavior transition probability data 28A, and the behavioral time probability distribution data 28E, and the current behavior 62 of the specific person.

The information extraction unit 44 of the present embodiment further extracts the elapsed time probability distribution data 28B, and the behavior prediction unit 46 predicts the future behavior of the specific person on the basis of the elapsed time probability distribution data 28B.

As described above, the behavior prediction device 10 of the present embodiment predicts the future behavior of the specific person in consideration of the behavioral time for each type of behavior. This makes it possible to consider tendency of the behavioral time, and thus it is possible to cope with a time shift in a case where the start time and the end time are shifted while the behavioral time is unchanged or in a case where only the time is shifted while a combination of the types of behavior to transition to the next behavior is unchanged. It is also possible to cope with a specific start time and a specific end time of a behavior whose type of behavior has a periodic occurrence time such as work or lunch.

Therefore, the behavior prediction device 10 of the present embodiment can predict the future behavior of the specific person with higher accuracy.

Although the mode in which the behavior prediction device 10 of the present embodiment predicts the future behavior of the specific person by using the end time probability distribution data 28D has been described, a mode in which the future behavior of the specific person is predicted without using the end time probability distribution data 28D may be adopted. For example, the behavior prediction device 10 may predict the end time from the start time probability distribution data 28C and the behavioral time probability distribution data 28E and predict the future behavior of the specific person by using the predicted end time.

In the present embodiment, the mode in which the specific person's behavior is estimated by using the behavior estimation model 26 on the basis of a feature value extracted from detection results of the sensor group 12 has been described. However, the method of estimating the specific person's behavior is not limited to the present mode. For example, the specific person himself/herself or a user who observes the specific person's behavior may input the type of behavior of the specific person to the behavior prediction device 10. Further, for example, the behavior prediction device 10 may estimate the specific person's behavior from the start time probability distribution data 28C and time.

Various types of processing executed by the CPU reading software (program) in each of the above embodiments may be executed by various processors other than the CPU. Examples of the processors in this case include a programmable logic device (PLD) in which a circuit configuration can be changed after manufacturing, such as a field-programmable gate array (FPGA), and a dedicated electric circuit that is a processor having a circuit configuration exclusively designed for executing specific processing, such as an application specific integrated circuit (ASIC). The behavior prediction processing may be performed by one of those various processors or may be performed by a combination of two or more processors of the same type or different types (e.g. a plurality of FPGAs or a combination of a CPU and an FPGA). A hardware structure of those various processors is, more specifically, an electric circuit in which circuit elements such as semiconductor elements are combined.

In each of the above embodiments, the aspect in which the behavior prediction program 25A and the statistical information extraction program 25B each are stored (installed) in advance in the storage 24 has been described, but the present invention is not limited thereto. The behavior prediction program 25A and the statistical information extraction program 25B may each be provided in the form stored in a non-transitory storage medium such as a compact disk read only memory (CD-ROM), a digital versatile disk read only memory (DVD-ROM), or a universal serial bus (USB) memory. Further, the behavior prediction program 25A and the statistical information extraction program 25B may each be downloaded from an external device via a network.

Regarding the above embodiments, the following supplementary notes are further disclosed.

### (Supplementary Note 1)

A behavior prediction device including:
a memory; and
at least one processor connected to the memory, in which
the processor is configured to
extract start time probability distribution data indicating a probability distribution of a start time for each type of behavior, behavior transition probability data indicating a probability of transition from one behavior to the other behavior for each combination of the types of behaviors, and behavioral time data regarding a behavioral time of the each type of behavior on the basis of a series of past behaviors indicating a past behavior history of the specific person in chronological order, and
predict the future behavior of the specific person on the basis of the start time probability distribution data, the behavior transition probability data, the behavioral time data, and a current behavior of the specific person.

### (Supplementary Note 2)

A non-transitory storage medium storing a program executable by a computer to execute behavior prediction processing, in which
the behavior prediction processing
extracts start time probability distribution data indicating a probability distribution of a start time for each type of behavior, behavior transition probability data indicating a probability of transition from one behavior to the other behavior for each combination of the types of behaviors, and behavioral time data regarding a behavioral time of the each type of behavior on the basis of a series of past behaviors indicating a past behavior history of the specific person in chronological order, and
predicts the future behavior of the specific person on the basis of the start time probability distribution data, the behavior transition probability data, the behavioral time data, and a current behavior of the specific person.

Reference Signs List

| | |
|---|---|
| 10 | Behavior prediction device |
| 12 | Sensor group |
| 21 | CPU |
| 22 | ROM |
| 23 | RAM |
| 24 | Storage |
| 25A | Behavior prediction program |
| 25B | Statistical information extraction program |
| 26 | Behavior estimation model |
| 28 | Statistical information |
| 28A | Behavior transition probability data |
| 28B | Elapsed time probability distribution data |
| 28C | Start time probability distribution data |
| 28D | End time probability distribution data |
| 28E | Behavioral time probability distribution data |
| 30 | Display unit |
| 32 | Communication I/F |
| 39 | Bus |
| 40 | Feature value extraction unit |
| 42 | Behavior estimation unit |
| 44 | Information extraction unit |
| 46 | Behavior prediction unit |
| 48 | Occurring behavior/behavior start time prediction |
| | unit |
| 50 | Behavior end time prediction unit |
| 60 | Series of past behaviors |
| 62 | Current behavior |

1. A behavior prediction device that predicts future behavior of a specific person, the behavior prediction device comprising:
a memory; and
a processor configured to execute a process, the process including:
extracting start time probability distribution data indicating a probability distribution of a start time for each type of behavior, behavior transition probability data indicating a probability of transition from one behavior to the other behavior for each combination of the types of behaviors, and behavioral time data regarding a behavioral time of the each type of behavior on the basis of a series of past behaviors indicating a past behavior history of the specific person in chronological order; and
predicting the future behavior of the specific person on the basis of the start time probability distribution data, the behavior transition probability data, the behavioral time data, and a current behavior of the specific person.

2. The behavior prediction device according to claim 1, wherein
the behavioral time data includes behavioral time probability distribution data indicating a probability distribution of the behavioral time during which a behavior is performed for the each type of behavior and elapsed time probability distribution data indicating, for each type of next behavior, a probability distribution of elapsed time from start of one behavior to start of the other behavior for the each combination of the types of behaviors.

3. The behavior prediction device according to claim 1, further comprising:
estimating the current behavior of the specific person on the basis of a feature value obtained from a detection result of a sensor that detects an event that changes depending on a person's behavior.

4. The behavior prediction device according to claim 1, wherein:
extracting end time probability distribution data indicating a probability distribution of an end time for the each type of behavior on the basis of the series of past behaviors; and
predicting the future behavior of the specific person on the basis of the end time probability distribution data.

5. A behavior prediction method of predicting future behavior of a specific person, the method comprising:
extracting start time probability distribution data indicating a probability distribution of a start time for each type of behavior, behavior transition probability data indicating a probability of transition from one behavior to the other behavior for each combination of the types of behaviors, and behavioral time data regarding a behavioral time of the each type of behavior on the basis of a series of past behaviors indicating a past behavior history of the specific person in chronological order; and
predicting the future behavior of the specific person on the basis of the start time probability distribution data, the behavior transition probability data, the behavioral time data, and a current behavior of the specific person.

6. (canceled)

7. The behavior prediction device according to claim 3, wherein:
a feature extraction circuitry extracts features related to events that change due to the behavior of the specific person from the detection result of the sensor.

8. The behavior prediction device according to claim 7, wherein:
the feature extraction circuitry extracts vital sign, location, temperature, humidity, illuminance, sound volume, sleeping state, awake state, and electricity consumption of the specific person.

9. The behavior prediction method according to claim 5, wherein
the behavioral time data includes behavioral time probability distribution data indicating a probability distribution of the behavioral time during which a behavior is performed for the each type of behavior and elapsed time probability distribution data indicating, for each type of next behavior, a probability distribution of elapsed time from start of one behavior to start of the other behavior for the each combination of the types of behaviors.

10. The behavior prediction method according to claim 5, the method further comprising:
estimating the current behavior of the specific person on the basis of a feature value obtained from a detection result of a sensor that detects an event that changes depending on a person's behavior.

11. The behavior prediction device according to claim 5, the method further comprising:
extracting end time probability distribution data indicating a probability distribution of an end time for the each type of behavior on the basis of the series of past behaviors; and
predicting the future behavior of the specific person on the basis of the end time probability distribution data.

12. The behavior prediction method according to claim 10, wherein:
a feature extraction circuitry extracts features related to events that change due to the behavior of the specific person from the detection result of the sensor.

13. The behavior prediction method according to claim 12, wherein:
the feature extraction circuitry extracts vital sign, location, temperature, humidity, illuminance, sound volume, sleeping state, awake state, and electricity consumption of the specific person.

14. A computer-readable non-transitory recording medium storing computer-executable program instructions that when executed by a processor cause a computer to execute a behavior prediction method comprising:
extracting start time probability distribution data indicating a probability distribution of a start time for each type of behavior, behavior transition probability data indicating a probability of transition from one behavior to the other behavior for each combination of the types of behaviors, and behavioral time data regarding a behavioral time of the each type of behavior on the basis of a series of past behaviors indicating a past behavior history of the specific person in chronological order; and
predicting the future behavior of the specific person on the basis of the start time probability distribution data, the behavior transition probability data, the behavioral time data, and a current behavior of the specific person.

15. The computer-readable non-transitory recording medium according to claim 14 wherein the behavior prediction method further comprises:
behavioral time probability distribution data indicating a probability distribution of the behavioral time during which a behavior is performed for the each type of behavior and elapsed time probability distribution data indicating, for each type of next behavior, a probability distribution of elapsed time from start of one behavior to start of the other behavior for the each combination of the types of behaviors.

16. The computer-readable non-transitory recording medium according to claim 14 wherein the behavior prediction method further comprises:
estimating the current behavior of the specific person on the basis of a feature value obtained from a detection result of a sensor that detects an event that changes depending on a person's behavior.

17. The computer-readable non-transitory recording medium according to claim 14 wherein the behavior prediction method further comprises:
extracting end time probability distribution data indicating a probability distribution of an end time for the each type of behavior on the basis of the series of past behaviors; and
predicting the future behavior of the specific person on the basis of the end time probability distribution data.

18. The computer-readable non-transitory recording medium according to claim 17 wherein the behavior prediction method further comprises:
extracting features related to events that change due to the behavior of the specific person from the detection result of the sensor.

19. The computer-readable non-transitory recording medium according to claim 18 wherein the behavior prediction method further comprises:
extracting vital sign, location, temperature, humidity, illuminance, sound volume, sleeping state, awake state, and electricity consumption of the specific person.

20. The behavior prediction device according to claim 1, wherein:
a behavior estimation model is obtained by training a learning model.

21. The behavior prediction device according to claim 20, wherein:
the behavior estimation model is further trained by inputting a training data, vectorizing extracted features, and outputting an estimation result.
